(19) United States
(12) Patent Application Publication
(10) Pub. No.: US 2006/0224123 A1
(43) Pub. Date: Oct. 5, 2006

(54) Device for administering a fluid product comprising optical scanning

(76) Inventors: Kurt Friedli, Lyssach (CH); Ulrich Haueter, Grosshoechstetten (CH); Fritz Kirchhofer, Sumiswald (CH)

Correspondence Address: DORSEY & WHITNEY LLP;INTELLECTUAL PROPERTY DEPARTMENT, SUITE 1500, 50 SOUTH SIXTH STREET, MINNEAPOLIS, MN, 55402-1498, US

(21) Appl. No.: 11/327,843

(22) Filed: Jan. 6, 2006

Related U.S. Application Data
(63) Continuation of application No. PCT/CH04/00397, filed on Jun. 25, 2004.

(30) Foreign Application Priority Data
Jul. 9, 2003 (DE) 103 30 985.3

(51) Int. Cl. A61M 5/00

(52) U.S. Cl. 604/207; 604/211

(57) ABSTRACT

A device for administering a substance including a measuring mechanism for measuring, without contact, a position, the measuring involving at least two elements of the device, at least one of the elements moveable with respect to the other, the measuring mechanism including at least two optical sensors for sensing a relative movement between the elements, the relative movement providing a profile trajectory. The present invention encompasses a method for measuring, without contact, a relative position between elements or structures which can be moved relative to each other, wherein sensors sense and/or record a profile trajectory associated with the elements or structures when one element is moved relative to another element, the trajectory processed to determine the position.

### CROSS-REFERENCE TO RELATED APPLICATION(S)

This application is a continuation of International Patent Application No. PCT/CH2004/000397, filed on Jun. 25, 2004, which claims priority to German Application No. DE 103 30 985.3, filed on Jul. 9, 2003, and the entire content of both applications is incorporated herein by reference.

### BACKGROUND

The present invention relates to devices for delivering, administering or dispensing substances, and to methods of making and using them. More particularly, it relates to a device for administering a fluid product, comprising measuring means for measuring, without contact, a position of elements of the administering device which can be moved with respect to each other, and to a method for measuring, without contact, their position with respect to each other. More particularly, in one embodiment, the present invention relates to measuring the setting of an administering or dosing device or mechanism of an injection apparatus.

Devices such as those that the present invention relates to are used in many areas, including in the medical field for administering or injecting a medical or pharmaceutical product. Injection apparatus, such as for instance injection pens, may be used for dispensing insulin, hormone preparations and the like. An injection apparatus comprises various mechanical means, such as an administering or dosing means, to be able to exactly dispense a particular product dosage from the apparatus. In order to monitor the administering process and its accuracy, it is usual to arrange sensors or probes within the apparatus, which detect the movement of various elements of the mechanical means. From this, the setting of the mechanical means is ascertained, for example by means of a microprocessor, and can be indicated on or by the injection apparatus by a mechanical or electronic display.

Since mechanical scanning is susceptible to contamination, moisture and wear and exhibits large tolerances between the individual elements, which restricts the accuracy in measuring the setting of an injection apparatus, non-contact methods for determining the setting of such an apparatus have been developed. To this end, a number of sensors or measuring devices may be arranged at various points on the apparatus.

WO 02/064196 A1 discloses an injection apparatus controlled by a closed switch unit comprising integrated sensors which monitor selected parameters of the apparatus. The closed switch unit is fixed within the injection apparatus. At least two pairs of integrated Hall elements are used as the sensors. The Hall elements co-operate with a magnetised ring which alternately exhibits north and south poles. The ring is arranged within a dosing means and is moved around the longitudinal axis of the injection apparatus in accordance with a rotational movement for setting a product dosage. In order to measure the volume of a dosage setting, it is necessary to determine the rotational movement of the magnetic ring relative to the closed switch unit. To this end, the Hall elements are arranged on a circular arc opposing the magnetic ring, in a defined arrangement with respect to each other and the magnetic ring. When movement is started, a start angle is defined and, on the basis of measuring the magnetic field during the movement of the magnetic ring relative to the Hall elements, an end angle is determined once the movement is terminated. The start and end angles and the measured magnetic field are compared with a stored table and a product dosage set is determined from the comparison.

EP 1095668 discloses an electronic administering pen for medical purposes which, in order to measure the setting of an administering means of the pen, measures the linear position of a helical rod of the administering mechanism or the rotational position of a setting button of a dosing means. An optical code converter comprising a code disc coupled to the rotational movement of the setting button is used. The rotational movement of the code disc is measured by an optical receiver. A microprocessor converts the number of rotations by the code disc into a dosage amount corresponding to the setting. Another sensor is provided between the windings of the helical rod of the administering means and registers the movement in the longitudinal direction along the longitudinal axis of the pen. The administered amount of a product is determined from the shift of the helical rod. The two sensors operate independently of each other and each determine only one movement direction of a mechanical means of the pen.

While such measuring means for measuring without contact can increase the accuracy in measuring a setting as compared to mechanical scanning, the arrangement of the individual parts of such measuring means within the apparatus is complex, such that manufacturing the apparatus is complicated and expensive. In addition, the circuitry and measuring methods of the aforementioned measuring means are susceptible to moisture, vibrations and other such effects. Accommodating the individual parts of the measuring means, such as the sensors and the counter pieces for the sensors, often requires structural changes in an administering device, making it unnecessarily large or even restricting the other mechanisms and/or functions of the device.

### SUMMARY

It is an object of the present invention to provide a device for administering a fluid product incorporating measuring means for measuring and/or assessing operational parameters of the device. It is another object of the present invention to provide measuring means for use with devices for administering, delivering, injecting or infusion a substance, and a method of making and using the measuring means. Another object of the present invention is to reduce the number of components needed to accomplish the aforementioned objects, and to enable only small movements of the elements of the device (e.g., operational, mechanical, functional structures or features of the device) to be measured or assessed exactly or as precisely as possible. It is another object of the present invention to provide a non-contact method for measuring the setting and/or performance of mechanical means of an administering device, said method enabling a movement and the position of selected elements of the apparatus to be easily determined and increasing the accuracy in measuring the setting and/or performance.

In one embodiment, the present invention comprises a device for administering a substance comprising a measuring mechanism for measuring and/or assessing, without contact, a position relative to at least two elements of the device, at least one of the elements moveable with respect to the other, the measuring mechanism comprising at least two optical sensors for sensing a relative movement between the elements, the relative movement providing a profile trajectory. The present invention encompasses a method for measuring, without contact, a relative position between elements or structures which can be moved relative to each other, wherein sensors sense and/or record a profile trajectory associated with the elements or structures when one element is moved relative to another element, the trajectory processed to determine the position.

The present invention involves administering, delivering or dispensing devices such as injection devices. Typically, injection devices comprise various mechanical means such as an administering or dosing means constructed from a number of elements at least some of which are moved relative to each other within the apparatus when the apparatus is operated. For example, to administer a product from the apparatus, a sliding element such as a toothed rod is moved along the longitudinal axis of the apparatus relative to a product container, an apparatus casing or other guiding elements of the administering means. A dosing means for setting a dosage volume to be administered may include a rotational element which is rotated relative to the casing or a threaded rod. In accordance with the present invention, in addition to these and/or other operational or functional structures or mechanisms, the injection apparatus comprises a measuring means which measures the setting of a mechanism of the apparatus and, therefore, of the setting or state of the injection apparatus, by determining the movement of elements relative to each other.

In accordance with the present invention, in one embodiment, the measuring means includes at least two optical sensors. The optical sensors can be provided by suitable optoelectronic units using which optical radiation can be generated, detected, transmitted, converted into electrical signals and processed. An optical sensor can therefore consist for example of radiation emitters, radiation receivers or optocouplers. In some preferred embodiments, the optical sensors may comprise a laser detector, reflex detector or light barrier.

In some embodiments, the at least two optical sensors are arranged, fixed with respect to each other, on at least a first element of an injection apparatus. The two sensors are therefore in a fixed spatial relationship to each other. It is possible for the sensors to be fixed to different elements of the apparatus, which for their part are fixed with respect to each other. The at least two optical sensors are arranged on the first element such that they oppose a second element of the injection apparatus. It is not necessary to pay any particular consideration to the distance between a first element, i.e., a sensor, and a second element. Care should merely be taken that no other elements lie between the first element and the second element, which could disrupt optical measuring.

The measuring means or measuring means component on the second element also exhibits a surface profile which provides a different predetermined profile trajectory for each of the sensors when the first element and the second element are moved relative to each other. The surface structure of the second element therefore exhibits a characteristic formation or an additional agent is provided which gives the second element a characteristic surface structure. When the first element is moved relative to the second element, such as when a sliding element of a dosing or administering means is slid or a rotational element rotated, relative to the first element comprising the sensors, the surface profile of the second element is guided past the sensors and the sensors measure the profile trajectory of the surface profile, wherein the surface profile is formed such that the sensors each register one predetermined profile trajectory and the profile trajectory measured by one sensor during movement differs from the profile trajectory measured by another sensor during this movement.

The surface profile preferably comprises a profile area or of a number of profile areas exhibiting a periodic surface structure running in the movement direction of the elements. In a surface profile comprising only one profile area of a periodic surface structure, the sensors are offset in the movement direction and arranged at different points of the period of the surface structure. The sensors may be, for example, arranged adjacently in the movement direction, such that one sensor opposes a period maximum and one sensor for example opposes a period cusp point of the surface structure. In some preferred embodiments, however, the sensors are not both arranged opposite an extreme point of the period such as a maximum or minimum.

If the surface profile exhibits a number of profile areas comprising a periodic surface structure, the sensors can be arranged adjacently, transverse with respect to the movement direction, each over one profile area. A surface profile may comprise two homogeneous profile areas offset with respect to each other in the movement direction. Two sensors arranged adjacently, transverse with respect to the movement direction, therefore detect a particular period point such as for instance a period maximum of a profile area at different times when the second element is moved relative to the first element.

The periodic surface structure of a profile area can, for example, be created by at least two periodically alternating height levels. When the elements are moved relative to each other, the distance between a sensor and the surface of the second element therefore changes periodically in accordance with the alternating height levels. A simple cam shaft or cam disc may used to this end. A profile area of a surface profile of the second element could also be formed by periodically arranged holes or cavities on the surface. When the elements are moved relative to each other, the light beam of a radiation emitter of an optical sensor then either passes through the holes or cavities or can be reflected by the surface. The holes or cavities on the surface profile of the second element may, for example, be formed by one or more perforated or slit discs fixed on the second element.

It is also possible to form the profile areas of the surface profile using periodically alternating light and dark fields. This could, for example, be provided by coloring the second element or by an additional ring or strip on the second element. A light beam of a radiation emitter is absorbed and/or reflected differently by the light and dark fields. On the second element, the periodic surface structure of a profile area extends in the circumferential direction or in the longitudinal direction of a longitudinal axis of the injection apparatus. In some preferred embodiments, the surface profile of the second element comprises profile areas whose periodic surface structure extends both in the longitudinal direction and the circumferential direction of the injection apparatus.

A particular surface profile may be selected in view of the type of an optical sensor used. If a laser detector is used, the optical sensor measures the predetermined profile trajectory when the elements are moved relative to each other, for example by scanning height levels which periodically change during movement or the changing distance between the sensor and the surface of the second element. If a reflex detector is used as the optical sensor, the intensity of the light reflected by the surface profile is generally measured. The intensity changes as the elements are moved relative to each other, for example by a periodically changing distance between the surface of the second element and the sensor due to changing height levels of a profile area.

It is also possible to generate a change in intensity at the sensor using different angular positions of the surface of the second element with respect to the sensor, such that a light beam of the detector incident onto the surface is reflected in different directions in accordance with the predetermined surface profile. A profile area can then be formed by various faces running or extending obliquely with respect to the incident direction of the light. The faces of the surface profile are then also arranged obliquely with respect to the longitudinal axis of the injection apparatus.

It is also possible, when using a reflex detector, to generate a predetermined profile trajectory by the light beam being reflected more or less by light and dark fields of the surface profile. If a light barrier is used, the predetermined profile trajectory can be generated by periodically arranging holes or cavities, for example on a perforated or slit disc.

When configuring the profile areas of the surface profile, it is also possible to provide—in additional to the periodic surface structure—a reference point which is distinguished from the periodic surface structure. This can be accomplished, for examples, by a providing a particularly high or low height level, by providing a particularly large or narrow hole on a perforated disc, or by a face having an angular position with respect to the sensor which is different to the other faces.

The profile area recorded by each sensor is transmitted as a measurement signal to a microprocessor in the injection apparatus, which processes the individual measurement signals and ascertains from them the position of the first element and the second element with respect to each other. A dosage setting or an administered product amount can then be calculated from this newly ascertained position and the initial position before the elements were moved with respect to each other or another reference position. To this end, the initial position before movement is preferably stored in a memory and the newly calculated position is also stored in the memory as a new initial position. The ascertained data of the dosage setting or the product amount can be read from an optical display.

In one preferred embodiment of an injection apparatus in accordance with the invention, two optical sensors are arranged on a first element which is fixed relative to a casing of the injection apparatus. The second element is formed by a sliding element which can be shifted in the longitudinal direction of a longitudinal axis of the apparatus, relative to the casing, or by a rotational element which can be rotated around the longitudinal axis of the apparatus, relative to the casing, as described above for an administering or dosing means.

In some embodiments, when determining the setting of the first element and the second element with respect to each other, it is also possible to measure discrete setting positions. A discrete setting position may, for example, correspond to a period or half a period of the periodic surface structure of a profile area. It is then particularly advantageous if, in accordance with the discrete setting positions for a movement direction on a second element, a surface profile which is suitable for measuring another movement direction is provided. If, for example, a number of discrete setting positions are determined on the circumference of a rotational element, it is possible to provide the surface profile, in accordance with these discrete rotational positions on a sliding element, using a number of homogeneous profile area combinations having a periodic surface structure in the longitudinal direction of the apparatus. A surface profile area is then assigned to each discrete rotational position, said surface profile area enabling a movement in the longitudinal direction of the sliding element to be measured, for example after the rotational movement of the rotational element has been measured. It is then advantageous if the two sensors oppose both the rotational element and the sliding element and therefore also the corresponding profile area of the rotational element and of the sliding element. The rotational element and the sliding element may be formed by a single element which can be both rotated and shifted relative to the first element. This can be provided by a sleeve of the apparatus which is rotated around the longitudinal axis of the apparatus in order to set the dosage and is shifted relative to the first element in order to administer the product from the apparatus.

It is conceivable to provide, in addition to the two optical sensors, a third optical sensor which serves as a monitor switch for the two optical sensors. A third optical sensor can improve the reliability of the injection apparatus. In one embodiment, the surface profile for the third optical sensor can be formed such that it registers a change in surface every time either the first sensor or the second sensor records a change. If the third sensor registers a change in surface and neither of the two other sensors records a change, then the injection apparatus is operating incorrectly.

Using optical sensors increases the design possibilities in the interior of an administering device, since the distance between an optical sensor and the surface profile necessary for measuring is very flexible. Optical sensors are typically small components or devices, such that the size of an administering device can be reduced. In most cases, the optical sensors are available as standard components, which makes the device cost-effective to manufacture. By combining at least two optical sensors and adapting the surface profile which co-operates with the sensors, it is possible to accurately and reliably determine the setting of two elements with respect to each other.

The present invention encompasses a method for measuring, without contact, a position between elements of a device for administering a fluid product, in particular an injection apparatus, wherein said elements can be moved relative to each other. In one embodiment, the method involves an apparatus comprising at least two optical sensors which are fixed with respect to each other and arranged on at least a first element and oppose a surface profile on a second element which can be moved with respect to the first element. Accordingly, an injection apparatus such as described above may be involved. In one embodiment, the method is used in an injection apparatus including an administering means comprising a sliding element which can be moved in the longitudinal direction of the longitudinal axis of the apparatus, and including a dosing means comprising a rotational element which can be rotated around the longitudinal axis. Furthermore, in one embodiment, an element which is fixed with respect to a casing of the injection apparatus, or the casing itself, is preferably used as the first element.

In accordance with the invention, each of the optical sensors is moved over the surface profile of the second element when the first element is moved relative to the second element, each sensing and/or recording a different predetermined profile trajectory. The profile trajectories recorded by each of the sensors are processed together, in order to determine the path distance travelled during movement. For a sliding element of an administering means, this path distance can correspond to the advance of a piston, which determines a product amount administered from the apparatus. For a rotational element of a dosing means, the path distance travelled corresponds to an angular distance using which the change in a dosage setting can be indicated. In principle, it is possible to determine a path distance travelled using only one sensor. By processing the different profile trajectories of various sensors, however, the path distance travelled can be determined reliably and substantially continuously in fine gradations, even when the periodicity of an individual surface area cannot allow measuring to be so finely gradated. In order to ascertain the position of the first element with respect to the second element, the profile trajectories recorded by the sensors are outputted as measurement signals to a suitable microprocessor or computer, and the path distance travelled is correlated with an initial position before the movement is started or with a reference position, as explained above.

A surface profile of the second element comprises one or more profile areas having a predetermined periodic surface structure, such as, for example, described above. In order to record a predetermined profile trajectory, the optical sensors are guided over a profile area of the surface profile when the elements are moved relative to each other. A light beam of an optical sensor is then differently affected in accordance with the periodic surface structure of the profile area, which creates the predetermined profile trajectory during movement. The optical sensors can record a different predetermined profile trajectory by being arranged over or operatively associated with the same profile area or over various profile areas, as described above. If the optical sensors are guided over the profile areas or the profile areas are moved past the optical sensors, a characteristic point of the periodic surface structure is recorded, offset in time, by the optical sensors. Such a characteristic point can be formed by the edge of changing height levels or by the start of a hole or cavity.

In may not be possible to form the periodic surface structure of a profile area of the surface profile as closely or finely as desired. The shortest possible path distance which may then be measured is therefore determined by the periodic surface structure. For a periodic surface structure consisting of two periodically alternating height levels, the minimum unit of distance which can be measured is, for example, given by the distance between two edges of the height level transitions. For a perforated disc, the minimum distance which can be measured is defined by the distance between the holes. In the method in accordance with the invention, different predetermined profile trajectories are recorded by the sensors when the elements are moved relative to each other, and processed together. This enables distances to also be determined which are shorter than the minimum path distance which can be measured by a sensor, since a characteristic point of a profile trajectory recorded by another sensor can lie within the minimum path distance which can be measured by a sensor.

It is advantageous to also be able to easily determine the movement direction of the elements with respect to each other by processing the different profile trajectories. If, for example, a surface profile consists of a first and second profile area arranged adjacently and exhibiting the same periodic surface structure in the form of periodically changing steps, and of two sensors which are adjacently arranged transverse with respect to the movement direction, each over a profile area, then the edge of a step of the profile areas is first registered by the one sensor or the other sensor, depending on the movement direction. The movement direction of the elements with respect to each other can easily be determined from such a characteristic relationship of the different profile trajectories measured by the sensors.

In one embodiment, the present invention comprises a device for administering a fluid product, such as for instance an injection apparatus, comprising a measuring means for measuring, without contact, a position between elements which can be moved with respect to each other, said measuring means including an optical sensor on a first element, said optical sensor facing a second element which can be moved with respect to the first element. The first element and the second element of the injection apparatus can be moved in the radial direction with respect to the longitudinal axis of the injection apparatus, such that the distance between the first element and the second element changes.

The optical sensor is in turn arranged on a first element which is fixed relative to a casing of the injection apparatus or is arranged on the casing itself. The second element can be a slider or a reset ring of a locking means of the injection apparatus, which in a first position unlocks the apparatus and in a second position, offset in the radial direction of the longitudinal axis with respect to the first position, locks the apparatus.

The optical sensor is therefore arranged generally opposite the second element, such that it can measure the changing distance between the first and the second element when the elements are moved relative to each other. When the elements are moved, a light beam of the optical sensor is differently deflected or reflected by a surface of the second element opposite the sensor, in accordance with changing distance, and this difference is registered by the sensor.

It is possible in principle for the element which can be moved radially with respect to the longitudinal axis to simultaneously also be able to be moved along the longitudinal axis or around the longitudinal axis. It is then advantageous if the surface opposite the first element exhibits a surface profile comprising one or more profile areas which are characteristic of various rotational or longitudinal positions. To this end, the surface profile can comprise various steps or a surface which extends obliquely with respect to the radial movement direction. In this way, the optical sensor can simultaneously determine a longitudinal position, a rotational position and a radial position.

The present invention enables the setting of an injection apparatus to be measured and/or assessed as optimally as possible by using optical sensors and formed surfaces which co-operate with the sensors. It is, of course, possible to combine different types and numbers of sensors and to measure the position of different pairs of first and second elements. The measurement signals of the various sensors or the ascertained settings of pairs of elements can then in turn be processed to help accurately monitor the injection apparatus. Advantageously, the sensors are arranged and the surface profiles formed such that a number of elements or movement directions can be measured using only a few sensors.

### BRIEF DESCRIPTION OF THE DRAWINGS

FIG. 1 is a longitudinal section through an area of an injection apparatus comprising a measuring means in accordance with the present invention using laser scanning, in accordance with one embodiment of the present invention;

FIG. 2 is a sectional schematic representation through a rotational element of the injection device from FIG. 1;

FIG. 3, including FIGS. 3a and b, is a longitudinal section through the area of the injection apparatus comprising a locking means, in accordance with the first embodiment;

FIG. 4, including FIGS. 4a and b is a cross-section through the area of the injection apparatus from FIG. 3 comprising an element which can be radially shifted, in a first and second position;

FIG. 5 is a longitudinal section through an area of an injection apparatus comprising a measuring means using reflection scanning, in accordance with another embodiment of the present invention;

FIG. 6 is a schematic representation as a cross-section through a rotational element from FIG. 5;

FIG. 7, including FIGS. 7a and b, is a longitudinal section through a locking means of the injection apparatus in an unlocked and a locked position, in accordance with the embodiment of FIG. 5;

FIG. 8 is a longitudinal section through an area of an injection apparatus comprising a measuring means using light barrier scanning, in accordance with another embodiment of the present invention;

FIG. 9 is a schematic representation as a cross-section through a rotational element from FIG. 8; and

FIG. 10, including FIGS. 10a and b, is a longitudinal section through a locking means of the injection apparatus of FIG. 8 in an unlocked and a locked position.

### DETAILED DESCRIPTION OF THE DRAWINGS

With regard to fastening, mounting, attaching or connecting the components of embodiments of the present invention, unless specifically described as otherwise, conventional fasteners such as screws, rivets, toggles, pins and the like may be used. Other fastening or attachment means appropriate for connecting components include friction fitting, adhesives, welding and soldering, the latter particularly with regard to electrical or processing components or systems. Any suitable electronic, electrical, communication, control or controller, computer or processing components may be used, including any suitable electrical components and circuitry, wires, wireless components, sensors, chips, boards, micro-processing or control system components, software, firmware, hardware, etc.

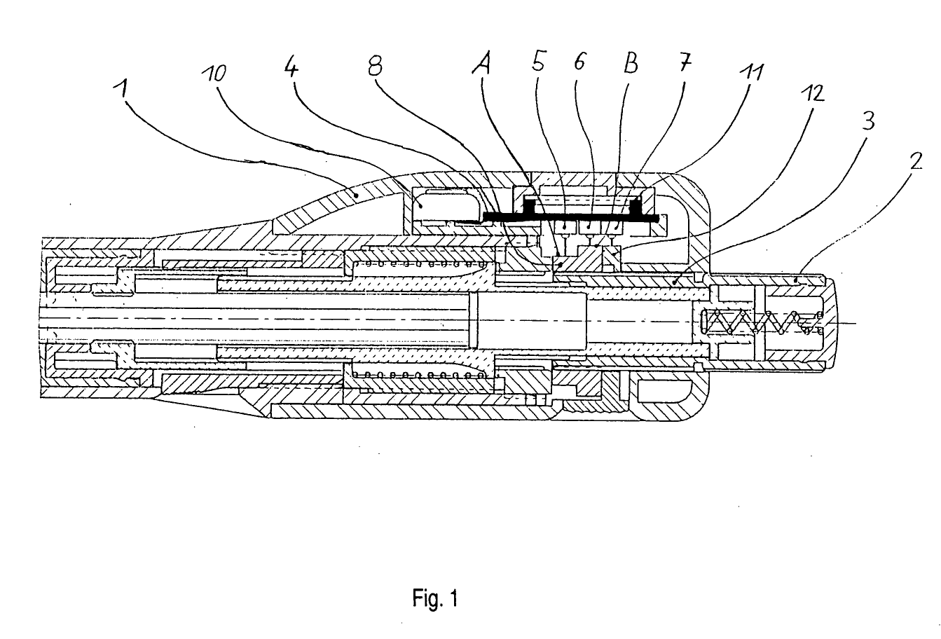

FIG. 1 shows one embodiment of the present invention in the form of an injection apparatus. The injection apparatus comprises a casing 1 in which a dosing means and an administering means are accommodated. The dosing means comprises a dosing button 2 which protrudes out of the casing 1. In its extension within the casing 1, the dosing button 2 comprises a sleeve 3 which transmits a rotational movement of the dosing button 2 onto the dosing means in order to set a dosage, wherein the sleeve 3 is moved within the casing 1 around the longitudinal axis of the injection apparatus and relative to the casing 1. The dosing button 2 can be pressed into the casing 1 in order to administer a product dosage, wherein the sleeve 3 is advanced in the longitudinal direction of the longitudinal axis of the injection apparatus and is moved in the longitudinal direction with respect to the casing 1. By pressing the dosing button 2 in, a product dosage is administered from the injection apparatus. In FIG. 1, the administering means is shown with various other elements which are not, however, described in more detail. In order to illustrate the invention, determining the setting of the sleeve 3 relative to the casing 1 shall be described, by way of example, for other elements which can be moved with respect to each other, wherein the casing 1 may be regarded as the first element and the sleeve 3 as the second element.

A beam or narrow plate 4 is fixed to the casing 1 which forms a part of the casing 1 and to which three optical sensors are attached in the form of laser detectors 5, 6 and 7. The laser detectors are attached adjacently in the longitudinal direction of the injection apparatus. A surface profile 8 comprising a first profile area A and a second profile area B is provided on the sleeve 3, opposite the sensors 5 and 6. The profile areas A and B exhibit a periodic surface structure in the form of two different alternating height levels. To this end, steps of equal length in the circumferential direction are arranged on a disc placed on the sleeve 3 and are repeated after a particular distance. The disc is coupled to the rotational movement of the sleeve 3, but remains at rest when the sleeve 3 is moved in the longitudinal direction. As may be gathered from FIG. 1, the laser detector 5 is arranged opposite the first profile area A and scans it with a light beam. The laser detector 6 is also arranged opposite the second profile area B and likewise scans it with a light beam.

The measurement signals of the laser detectors 5, 6 and 7 are forwarded to a microprocessor 10 for processing, said microprocessor 10 ascertaining the position of the sleeve 3 relative to the casing 1 from the measured data and converting it into, for example, a dosage setting value or administering value. The ascertained values may be indicated or displayed, for example on a display 11 below a transparent area of the casing 1.

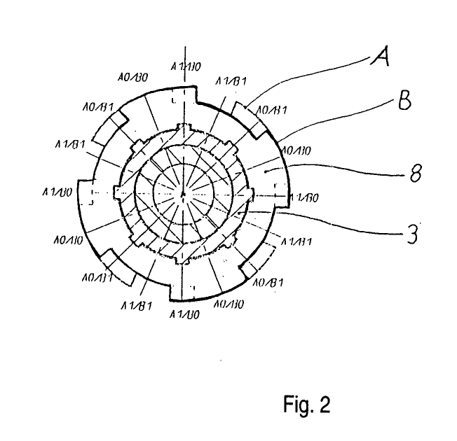

FIG. 2 shows a schematic section through the area of the sleeve 3 comprising the surface profile in accordance with the invention, where the second profile area B with its stepped shape can be seen in the foreground as a continuous line. The steps of the first profile area A are shown by the continuous lines which are offset to the left from the step trajectory of the second profile area B and by the broken lines within the steps of the second profile area B. It follows from this that in FIG. 2, the first profile area A is arranged offset anti-clockwise relative to the second profile area B in the circumferential direction, i.e., in the movement direction of the sleeve 3 relative to the casing 1. It may also be gathered from FIG. 2 that an edge of a step of the first profile area A does not lie in the middle of a step face of the profile area B. This avoids the sensor 5 and the sensor 6 simultaneously recording the change in a height level by scanning an edge of the steps both on the first profile area A and on the second profile area B when the sleeve 3 is moved relative to the casing 1.

FIG. 2 shows the sensors 5 and 6 scanning, at various settings of the surface profile 8 relative to the casing 1, wherein the designation A0 indicates that the sensor 5 registers a step on the first profile area A. At A1, a dip between the steps is registered. Correspondingly, the laser detector 6 registers a step of the second profile area B at a position B0 and a dip between the steps of the second profile area B at the position B1. At a position A1/B1, the sleeve 3 for example assumes a position relative to the casing 1 in which both the laser detector 5 and the laser detector 6 register a dip. At a position A0/B0, both detectors measure a step of the first profile area A or second profile area B, respectively. In a position A0/B1, the laser detector 5 measures a step of the first profile area A and the laser detector 6 measures a dip of the second profile area B. When the sleeve 3 is moved relative to the casing 1 over a particular path distance, therefore, the laser detectors 5 and 6 measure a different predetermined profile trajectory, by recording the individual positions which are guided past them during movement. In this way, the movement direction of the sleeve 3 can for example be ascertained, since if the sleeve 3 is moved clockwise in FIG. 2, the laser detector 6 will first measure a step of the second profile area B and only shortly afterwards the laser detector 5 will measure a step of the first profile area A. If the sleeve 3 is moved anti-clockwise in FIG. 2, the laser detector 5 will first measure a step of the first profile area A and only then will the laser detector 6 measure a step of the second profile area B.

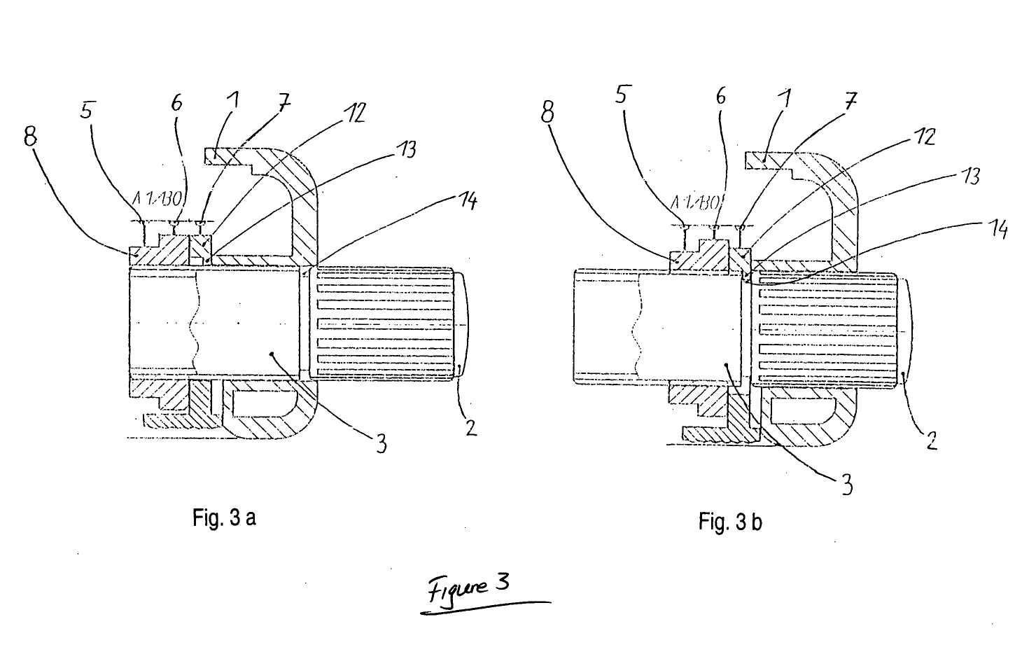

FIGS. 3a and 3b show a detail from FIG. 1, in which a locking means of the injection apparatus is shown, comprising a slider 12 which can be shifted relative to the casing 1 in a radial direction with respect to the longitudinal axis of the injection apparatus. To this end, the slider 12 is formed as an oval ring around the sleeve 3. FIG. 3a shows the slider 12 in an unlocked position in which it opposes the sleeve 3. FIG. 3b shows the slider 12 in a locked position in which the dosing button 2 is pressed into the casing 1, such that the sleeve 3 has been advanced in the longitudinal direction of the injection apparatus until a protrusion 13 of the slider 12 pointing towards the sleeve 3 engages with a groove 14 on the sleeve 3 and thus prevents the dosing button 2 from being pressed in further. The slider 12 is generally opposite the laser detector 7. In the unlocked position of FIG. 3a, a first distance is defined between the laser detector 7 and the surface of the slider 12 facing the detector. When the slider 12 is in its locked position, the protrusion 13 engages with the groove 14 and the distance from the surface of the slider 12 to the laser detector 7 increases. This change in distance is registered by the laser detector 7 and forwarded as a measurement signal to the microprocessor 10, which can then indicate the locking position on the display 11.

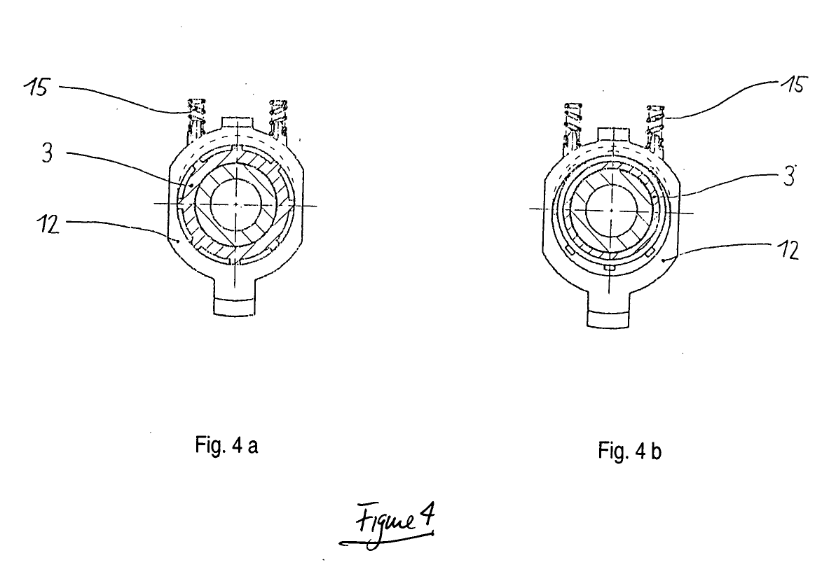

FIGS. 4a and 4b show a cross-section through the slider 12 of the locking means. FIG. 4a shows the slider 12 in an unlocked position in which already biased springs 15 act on it. An abutment for the springs 15 can for example be provided by the beam 4. When the dosing button 2 is pressed in, the sleeve 3 is shifted in the longitudinal direction until the protrusion 13 engages with the groove 14, as shown in FIG. 4b, wherein due to their bias, the springs 15 press the slider 12 into the groove 14, such that the slider 12 is moved in the radial direction both with respect to the casing 1 and with respect to the sleeve 3.

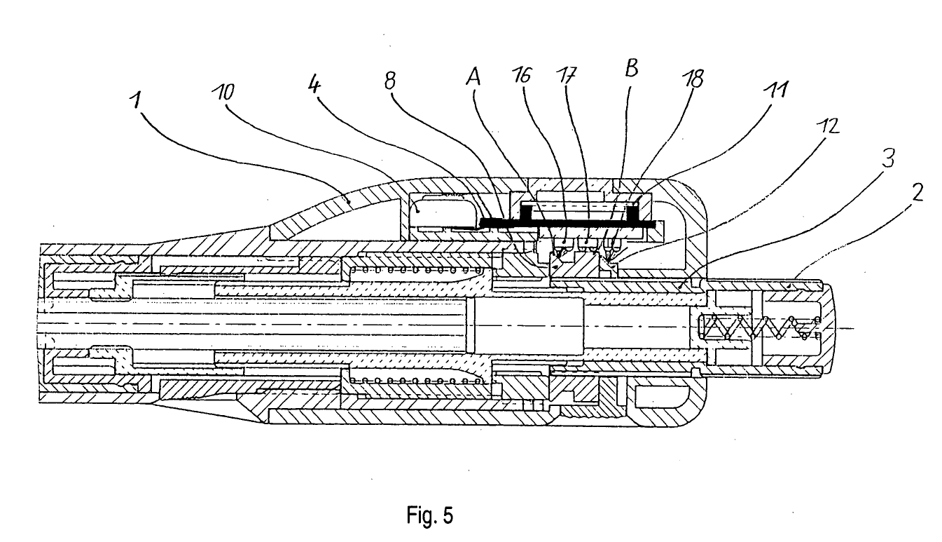

FIG. 5 shows another embodiment of the present invention, an injection apparatus comprising a measuring means in accordance with the present invention. In this embodiment, reflex detectors 16, 17 and 18 are fixed to the beam 4 of the casing 1 as the optical sensors. The reflex detectors 16, 17 and 18 include a radiation emitter and a radiation receiver which are arranged adjacently at a predetermined distance. As in the preceding exemplary embodiment, the surface profile 8 consists of a first profile area A and a second profile area B, wherein the first profile area A opposes the reflex detector 16 and the second profile area B opposes the reflex detector 17. In this embodiment, a step and a face extending obliquely between the steps alternate periodically on the profile areas A and B, wherein the oblique face slopes from one side of the profile area to the other, such that a light beam emitted by the radiation emitter encloses an angle with this oblique face such that it is reflected by the face onto the radiation receiver. If the sleeve 3 is rotated relative to the casing 1 in the circumferential direction of the injection apparatus, such that the profile areas A and B are guided past the reflex detectors 16 and 17, either a step or an oblique face opposes the detectors. The detectors 16 and 17 are arranged near enough to the surface profile 8 that a step of a profile area is guided tightly past it, while in the case of an oblique face, a small distance remains between the face and the detectors. If a step of a profile area opposes a detector 16 or 17, no emitted light is reflected to the radiation receiver. If a detector 16 or 17 opposes an oblique face, a light beam from the radiation emitter is reflected on the oblique face towards a radiation receiver which registers said light beam. In this way, the reflex detectors 16 and 17 can record a predetermined profile trajectory when the sleeve 3 is moved relative to the casing 1.

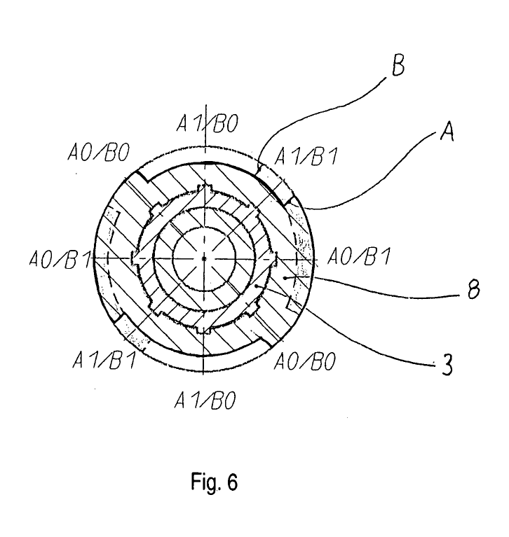

Comparable to FIG. 2, FIG. 6 shows various possible settings of the first profile area A and the second profile area B relative to the reflex detectors 16 and 17 which are adjacently arranged, transverse with respect to the movement direction. The first profile area A is shown in the foreground, in which an area hatched up to the edge represents a step of the first profile area A and an area left white represents an oblique face of the first profile area A. Behind the first profile area A, a second profile area B is shown in which the oblique faces are shown by the shaded areas and broken lines and the steps are shown by non-shaded areas. The two profile areas A and B are in turn arranged offset with respect to each other in the movement direction. As in FIG. 2, a number of possible settings of the profile areas A and B relative to the reflex detectors 16 and 17 are shown. The position A1/B0, for example, indicates that an oblique face of the first profile area A opposes the reflex detector 16 and a step of the second profile area B opposes the reflex detector 17. As in the preceding example embodiment, the reflex detectors 16 and 17 register a different predetermined profile trajectory when the surface profile 8 is guided past, due to the formation of the first profile area A and the second profile area B.

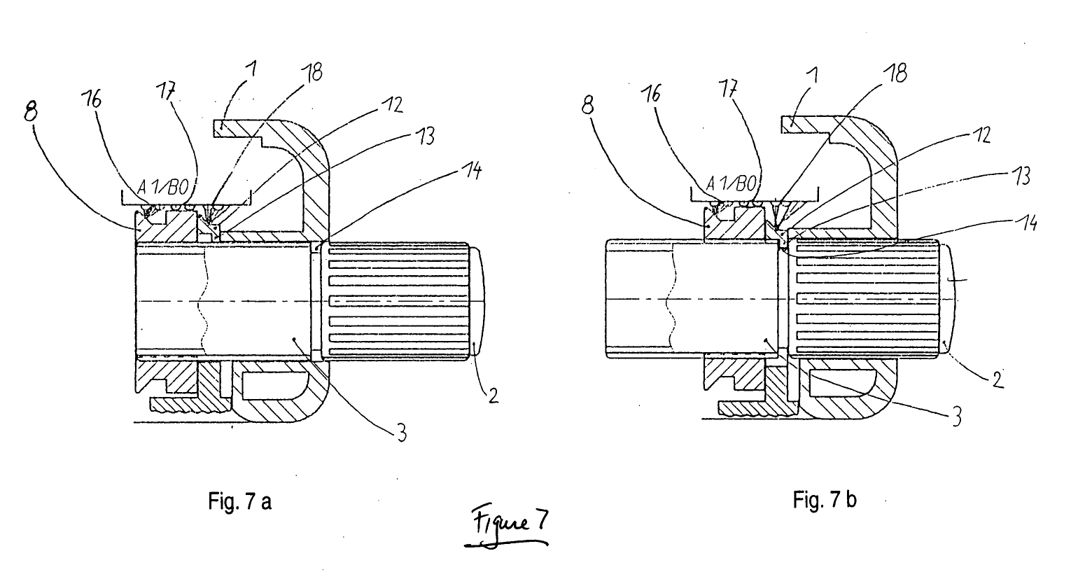

FIGS. 7a and 7b show an area from FIG. 5 comprising a locking means such as in FIGS. 3a and 3b. In this embodiment, the surface of the slider 12 opposite the reflex detector 18 is provided with an oblique face. In FIG. 7a, a light beam of the reflex detector is reflected on the oblique face such that it hits the radiation receiver of the detector. In FIG. 7b, the protrusion 13 of the slider 12 is locked into the groove 14 on the sleeve 3, which increases the distance between the surface of the slider 12 and the reflex detector 18. In this locked position, the light beam is reflected on the oblique face such that it does not hit the radiation receiver of the detector 18, but is rather guided past it. In this way, the reflex detector 18 can ascertain the radial setting of the slider 12 relative to the sleeve 3 or relative to the casing 1.

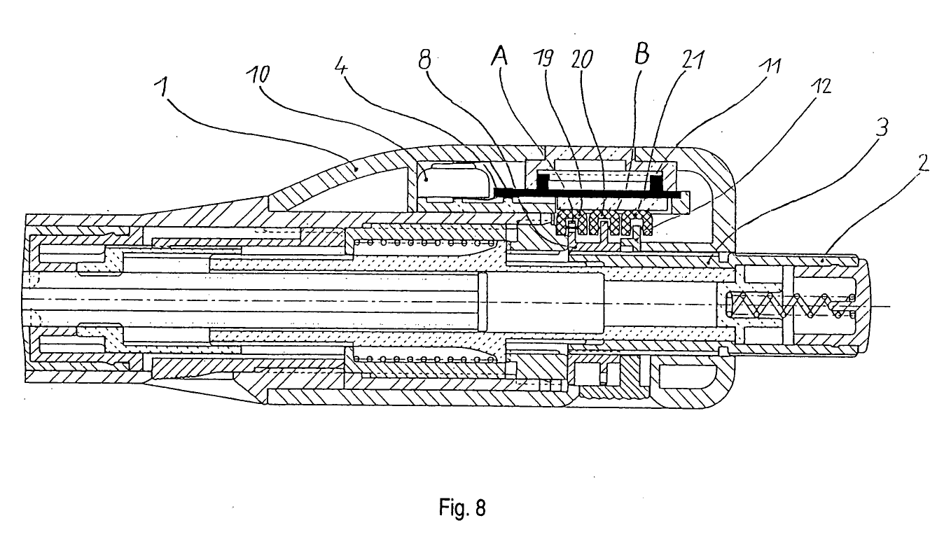

FIG. 8 shows another embodiment of the present invention, an injection apparatus comprising a measuring means in accordance with the present invention. Here, three optical sensors 19, 20 and 21 in the form of prong-shaped light barriers are attached to the beam 4 of the casing 1. One of the light barriers comprises two opposing arms, wherein one arm has a radiation emitter and the other arm has a radiation receiver. The surface profile 8 connected to the sleeve 3 is formed by a first perforated disc 22 as the first profile area A and a second perforated disc 23 as the second profile area B. The perforated disc 22 runs between the prong arms of the light barrier 19 and the perforated disc 23 runs between the prong arms of the light barrier 20. Holes are provided on the perforated discs 22 and 23 in a surface structure which is periodically repeated. If a hole comes to rest within a light barrier, the emitted light beam can be registered by the radiation receiver; if the disc face lies between the prongs, no light beam is registered.

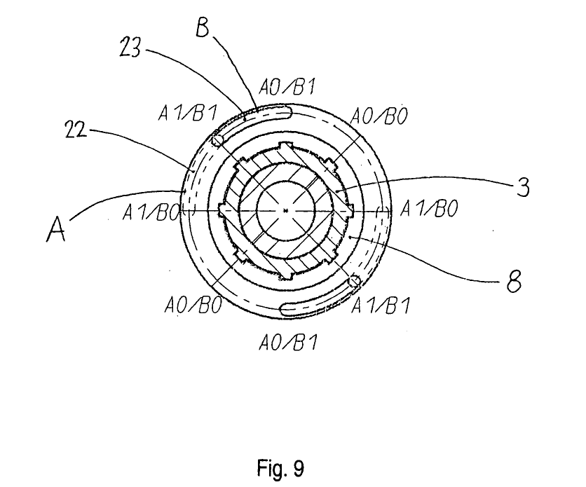

FIG. 9 schematically shows the arrangement of the two profile areas A and B or the two perforated discs 22 and 23 with respect to each other, in cross-section. The periodic surface structure of a profile area A or B is formed in the perforated discs by holes elongated in the circumferential direction, wherein in the foreground, the perforated disc 23 as the second profile area B is shown with holes which are shown as a continuous line. Behind it, the perforated disc 22 as the first profile area A is shown with holes which are indicated as broken lines. The perforated discs are offset with respect to each other in the movement direction, in order to provide a different profile trajectory for the two light barriers 19 and 20 when the sleeve 3 is moved relative to the casing 1. When arranging the perforated discs, care has been taken that symmetrical shifting does not arise, i.e., that a centre point of a hole of the profile area A does not come to rest on a centre point of the area between two holes of the profile area B. As in FIGS. 2 and 6, various possible settings of the perforated discs 22 and 23 relative to the light barriers 19 and 20 are shown. In the position A1/B0 for example, a hole is situated within the light barrier 19 and a disc wall is situated within the light barrier 20. By arranging the perforated discs 22 and 23 in this way, different predetermined profile trajectories can be recorded by the light barriers 19 and 20 when the sleeve 3 is moved.

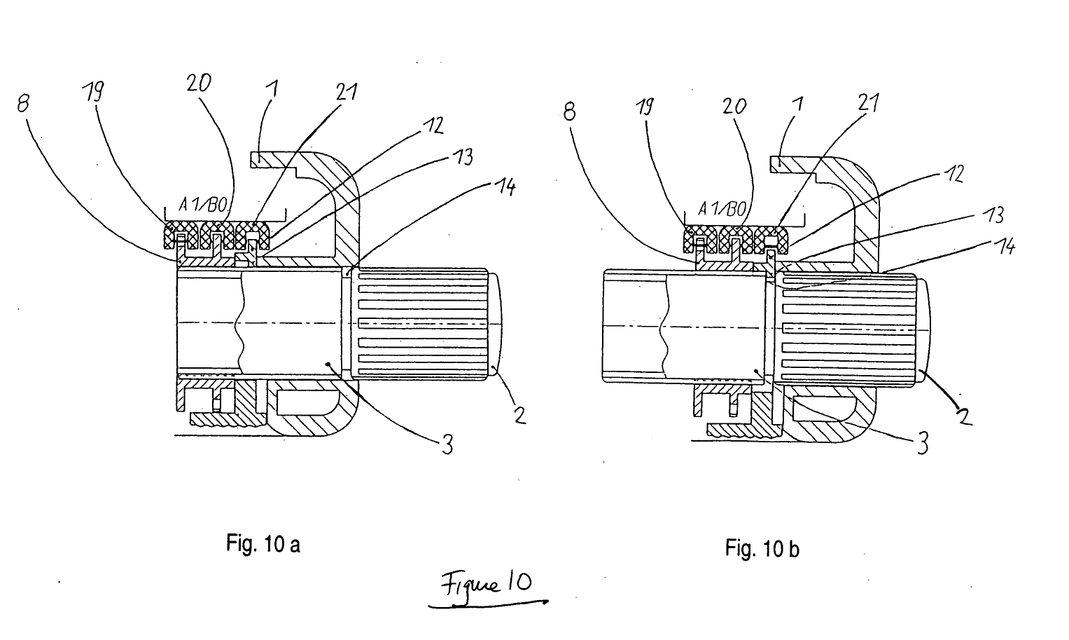

FIG. 10a shows an area of the injection apparatus comprising locking means. In this embodiment, the slider 12 comprises a protrusion 24 on its side opposite the light barrier 21, wherein in an unlocked position, said protrusion 24 engages between the prong arms of the light barrier 21, such that the radiation receiver does not register any light, as shown in FIG. 10a. In the locked position, in which the protrusion 13 of the slider 12 engages with the groove 14 on the sleeve 3, the distance between the slider 12 and the light barrier 21 in the radial direction increases, such that the protrusion 24 no longer engages between the prong arms of the light barrier 21, as shown in FIG. 10b. The light receiver can register the emitted light and therefore measure the locked position.

The invention has been explained in detail on the basis of the three exemplary embodiments. In principle, however, a multitude of different possible sensors and arrangements of sensors, including optical sensors, may be used relative to a selected surface profile without deviating from the concept of the invention. Thus, for example, it is possible to provide two co-operating optical sensors on opposite inner sides of a casing 1, or to combine different types of optical sensors. The described surface structures of the profile areas may also be combined. Thus, it is possible, when using reflex detectors, to additionally arrange light and dark fields on the oblique faces of the surface profile. The surface profiles described represent configurations of a surface profile which are cost-effective and easy to manufacture. No complicated secondary or additional treatment of, for example, simple injection-moulded parts, is necessary. It is also conceivable for a reset switch to use mechanical scanning in order to reduce the power consumption of the injection apparatus. As compared to a non-contact variant of the reset switch, in which the status of the apparatus is measured approximately every 1 to two milliseconds, the power consumption can be significantly reduced using a mechanical switch.

Embodiments of the present invention, including preferred embodiments, have been presented for the purpose of illustration and description. They are not intended to be exhaustive or to limit the invention to the precise forms and steps disclosed. Obvious modifications or variations are possible in light of the teachings herein. The embodiments were chosen and described to provide the best illustration of the principals of the invention and its practical application, and to enable one of ordinary skill in the art to utilize the invention in various embodiments and with various modifications as are suited to the particular use contemplated. All such modifications and variations are within the scope of the invention as determined by the appended claims when interpreted in accordance with the breadth they are fairly, legally, and equitably entitled.

1. A device for administering a fluid product, comprising a measuring means for measuring, without contact, a position between at least two elements of the administering device which can be moved relative to each other, wherein the measuring means comprising:
at least two optical sensors which are arranged fixed with respect to each other on at least a first element and opposite a second element which can be moved relative to the first element; and
a surface profile on the second element, which provides a different predetermined profile trajectory, which can be measured by the sensors, for each of the optical sensors when the at least first element and the second element are moved relative to each other.

2. The device for administering a fluid product as set forth in claim 1, wherein the surface profile consists of one profile area or a number of profile areas exhibiting a surface structure which is periodic in a movement direction.

3. The device for administering a fluid product as set forth in claim 2, wherein the optical sensors oppose the same profile area or various profile areas of the surface profile.

4. The device for administering a fluid product as set forth in claim 2, wherein the periodic surface structure of a profile area is provided by one of at least two periodically alternating height levels, periodically arranged holes or cavities and periodically alternating light and dark fields.

5. The device for administering a fluid product as set forth in claim 2, wherein the periodic surface structure on the second element extends in at least one of the circumferential direction and longitudinal direction of the administering device.

6. The device for administering a fluid product as set forth in claim 2, wherein a profile area comprises a reference point which is distinguished from the periodic surface structure of the profile area.

7. The device for administering a fluid product as set forth in claim 2, wherein a surface profile is formed from at least two homogeneous profile areas offset with respect to each other in the movement direction with respect to the periodic surface profile.

8. The device for administering a fluid product as set forth in claim 1, wherein the surface profile is provided by a cam disc or a perforated or slit disc which is coupled to the movement of the second element.

9. The device for administering a fluid product as set forth in claim 1, wherein an optical sensor is an optoelectronic unit in the form of a laser detector, a reflex detector or a light barrier.

10. The device for administering a fluid product as set forth in claim 1, wherein at least two optical sensors are arranged adjacently on a first element.

11. The device for administering a fluid product as set forth in claim 1, wherein the optical sensors are arranged on a first element which is formed by a casing or is fixed relative to the casing.

12. The device for administering a fluid product as set forth in claim 1, wherein one of the elements which can be moved is a sliding element which can be shifted in the longitudinal direction of the administering device relative to another element, or is a rotational element which can be rotated around the longitudinal axis of the administering device relative to another element.

13. The device for administering a fluid product as set forth in claim 1, wherein the at least two optical sensors oppose both a sliding element and a rotational element.

14. The device for administering a fluid product as set forth in claim 2, wherein discrete setting positions are determined in accordance with a period of the periodic surface structure of a profile area.

15. The device for administering a fluid product as set forth in claim 2, wherein a number of homogeneous surface profiles which oppose discrete rotational positions of a rotational element are provided along the longitudinal axis of the administering device on the circumference of a sliding element.

16. The device for administering a fluid product according to claim 1, further comprising at least a third sensor for monitoring the at least first sensor and second sensor.

17. A method for measuring, without contact, a position between elements of a device which can be moved relative to each other, said device for administering a fluid product and comprising at least two optical sensors fixed with respect to each other and arranged on at least a first element and opposed to a surface profile on a second element which can be moved with respect to the first element, wherein:
each of the optical sensors records a different predetermined profile trajectory of the surface profile when the at least first element is moved relative to the second element along the surface profile;
the profile trajectories recorded by each of the sensors are processed together, in order to determine a path distance travelled during movement; and
the path distance travelled is correlated with a reference position in order to ascertain the position of the first element and the second element with respect to each other.

18. The method as set forth in claim 17, wherein the movement direction of the first element and the second element with respect to each other is determined using the relationship of the different predetermined profile trajectories recorded by the sensors.

19. The method as set forth in claim 17, wherein the optical sensors record a predetermined profile trajectory by being guided over a profile area of the surface profile exhibiting a predetermined periodic surface structure, when the first element is moved relative to the second element.

20. The method as set forth in any one of claim 17, wherein the optical sensors record a different predetermined profile trajectory by being arranged at different period points over the same profile area, offset with respect to each other in the movement direction.

21. The method as set forth in any one of claim 17, wherein the optical sensors record a different predetermined profile trajectory by being arranged over various profile areas exhibiting different periodic surface structures.

22. The method as set forth in any one of claim 17, wherein the optical sensors record a different predetermined profile trajectory by being arranged at different period points over various profile areas exhibiting the same periodic surface structure offset with respect to each other.

23. The method as set forth in any one of claim 17, wherein the optical sensors record a different predetermined profile trajectory by recording a characteristic point of one or more profile areas, offset in time.

24. The method as set forth in any one of claim 17, wherein the position of the first element and the second element with respect to each other is determined as a discrete setting position in accordance with a periodic surface structure of a profile area.

25. The method as set forth in any one of claim 17, wherein before or after a discrete rotational position of a rotational element is measured, a movement in the longitudinal direction of the administering device is determined in the measured discrete rotational position.

26. A device for administering a fluid product, comprising a measuring means for measuring, without contact, a position between at least two elements of the administering device which can be moved relative to each other, wherein:
the measuring means comprises an optical sensor on a first element, said optical sensor the first element and the second element can be moved in the radial direction with respect to the longitudinal axis of the administering device, such that the distance between the first element and the second element changes when the elements are moved relative to each other.

27. The device for administering a fluid product as set forth in claim 26, wherein the optical sensor is arranged on a casing of the administering device or on a first element which is fixed relative to said casing.

28. The device for administering a fluid product as set forth in claim 26, wherein a surface of the second element facing the optical sensor exhibits a characteristic surface structure exhibiting a height profile which changes relative to the first element.

29. The device for administering a fluid product as set forth in any one of claim 28, wherein the surface of the second element exhibits a characteristic surface structure exhibiting a height profile in accordance with a rotational or longitudinal position of the second element with respect to the first element.

30. The device for administering a fluid product as set forth in any one of claim 29, wherein the second element is a slider of a locking means of the administering device.

31. A device for delivering a substance comprising:
a first element moveable during operation of the device and a second element; and
an optical sensor for sensing a relative movement between the elements, the relative movement providing a profile trajectory indicative of a condition associated with the device.

32. A method for measuring, without contact, a relative position between elements of a device which can be moved relative to each other, the method comprising the steps of:
sensing a relative movement between the elements;
calculating a profile trajectory stemming from the relative movement; and
using the profile trajectory to determine a condition of the device.

33. The method according to claim 32, wherein the sensing is performed by an optical sensor.

34. A device for administering a fluid product, comprising at least two elements at least one of which is moveable with respect to the other and a measuring means for measuring, without contact, a relative position of the at least two elements, the measuring means comprising:
at least two optical sensors which are arranged with respect to each other and a first element and a second element which can be moved relative to the first element; and
a different surface profile associated with each of said at least two elements, which, when at least one of the elements is moved relative to the other, provides a different predetermined profile trajectory, which can be sensed by the optical sensors.
